(19) United States
(12) Patent Application Publication
(10) Pub. No.: US 2022/0178922 A1
(43) Pub. Date: Jun. 9, 2022

(54) PATHOGEN SAMPLING AND TESTING

(71) Applicant: Merit Medical Systems, Inc., South Jordan, UT (US)

(72) Inventors: Fred Lampropoulos, Salt Lake City, UT (US); Ryan Davis, Centerville, UT (US); Geoffrey Russell, Ogden, UT (US); John Hellgeth, Salt Lake City, UT (US); Michael Dean Haslam, Sandy, UT (US); Shayna Bernice Judd, Magna, UT (US); Jim Mottola, West Jordan, UT (US)

(21) Appl. No.: 17/457,399

(22) Filed: Dec. 2, 2021

Related U.S. Application Data
(60) Provisional application No. 63/121,149, filed on Dec. 3, 2020.
(60) Provisional application No. 63/121,682, filed on Dec. 4, 2020.

(51) Int. Cl. G01N 33/569; G01N 33/543; A61B 10/00

(52) U.S. Cl. CPC G01N 33/56983; G01N 33/54388; A61B 2010/0087; G01N 2469/10; G01N 2333/165; A61B 10/00

(57) ABSTRACT

Devices and materials are disclosed for collecting breath samples and for testing such samples for the presence of a pathogen, for example the pathogen(s) associated with Coronavirus Disease 2019 (COVID-19). In some embodiments, a collection device can include a tube into which a subject can exhale or cough, and that provides for use of a filter to capture expired sample material. In some embodiments, a sample liquid can be created from the breath sample by addition of an indicator to render a pathogen in the sample readily detectable. In some embodiments, materials are provided for an assay to detect a pathogen in the sample liquid.

### RELATED APPLICATIONS

This application claims priority to U.S. Provisional Patent Application No. 63/121,149, filed on Dec. 3, 2020, entitled “PATHOGEN SAMPLING AND TESTING,” and United States Provisional Patent Application No. 63/121,682, filed on Dec. 4, 2020, entitled “PATHOGEN SAMPLING AND TESTING,” each of which is hereby incorporated herein by reference in its entirety.

### TECHNICAL FIELD

This application generally relates to devices, materials, and methods for collecting biological samples, particularly breath samples, and testing said samples for the presence of pathogens.

### BRIEF DESCRIPTION OF THE SEVERAL VIEWS OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a perspective view of a breath sample collection device in accordance with an embodiment.

FIG. 1B is a different perspective view of the breath sample collection device shown in FIG. 1A.

FIG. 2A is a perspective view of a breath sample collection device in accordance with another embodiment.

FIG. 2B is a different perspective view of the breath sample collection device shown in FIG. 2A.

FIG. 3 is a perspective view of a vial containing an immobilized indicator in accordance with an embodiment.

FIG. 4A is a perspective view illustrating a breath sample collection device and a vial arranged for coupling in accordance with one embodiment.

FIG. 4B is a perspective view of the collection device and vial of FIG. 4A in a coupled state and illustrates a use thereof in accordance with an embodiment.

FIG. 5 is a perspective view of a vial including a partition and containing an immobilized indicator in accordance with another embodiment.

FIG. 6A is a perspective view of an assay strip in accordance with an embodiment.

FIG. 6B is a perspective view of the assay strip of FIG. 6A showing a valid positive result of an assay of a sample.

FIG. 6C is a perspective view of the assay strip of FIG. 6A showing a valid negative result of an assay of a sample.

FIG. 7 illustrates a use of an assay strip and a vial of the present disclosure in accordance with an embodiment.

FIG. 8A is a perspective view of a system for detecting a pathogen in a breath sample in accordance with an embodiment.

FIG. 8B is another perspective view of the system of FIG. 8A.

FIG. 8C is an exploded view of the system of FIG. 8A.

FIG. 8D is another exploded view of the system of FIG. 8A.

FIG. 8E is a cross-sectional view of the system of FIG. 8A taken at the plane labeled as “8E”.

### DETAILED DESCRIPTION

Disclosed herein are devices and materials for collecting breath samples and for testing such samples for the presence of a pathogen, for example the pathogen(s) associated with Coronavirus Disease 2019 (COVID-19). In some embodiments, a collection device can include a tube into which a subject can exhale or cough, and that provides for use of a filter to capture expired sample material. In some embodiments, a sample liquid can be created from the breath sample by addition of an indicator to render a pathogen in the sample readily detectable. In some embodiments, materials are provided for an assay to detect a pathogen in the sample liquid.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

It will be appreciated that various features are sometimes grouped together in a single embodiment, figure, or description thereof for the purpose of streamlining the disclosure. Many of these features may be used alone and/or in combination with one another.

The directional terms “distal” and “proximal” are given their ordinary meaning in the art. That is, the distal end of a device for use on a subject means the end of the device furthest from the subject during use. The proximal end refers to the opposite end, or the end nearest the subject during use.

Current approaches to screening individuals for respiratory infections include sampling fluids collected from the distal airway. For example, samples of respiratory viruses are collected by (1) nasopharyngeal swabs that sample the back of the nasal passages or (2) saliva samples that are collected by expectoration into a sample container. However, since the locus of the infection of concern is the lungs, these sites only indirectly sample the viral loading in the respiratory tract. In addition, in both saliva and nasal mucus samples the pathogen of interest is presented in a complex matrix of other proteins that can interfere with analysis. The relative abundance of other components may mean that a given amount of sample material in fact provides a very small quantity of analyte to be detected by a test. Furthermore, often a significant amount of liquid is needed to extract a sufficient amount of the sample from collection devices such as nasopharyngeal swabs. Therefore, the problem of obtaining a sufficient amount of detectable analyte is exacerbated by dilution issues.

Exhaled breath can be an alternative sample material for detecting respiratory pathogens such as severe acute respiratory syndrome coronavirus 2 (SARS-CoV-2). Exhalation, and particularly coughing, by an infected individual can produce infectious aerosols, i.e., suspended particles containing pathogens, making expired breath an effective mode of disease transmission. It has been found that while humans produce infectious aerosols in a wide range of particle sizes, a predominance of pathogens in cough aerosols and from exhaled breath are in relatively small particles (<5 μm). The present disclosure discusses an approach to sampling virus aerosols more directly than is possible with current methods, and presents the sample in an aerosol form that is less complex to analyze.

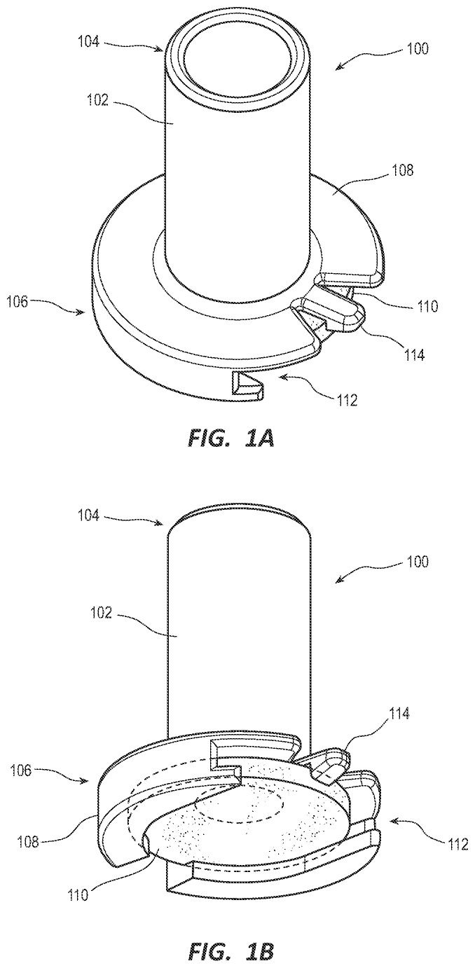

In an aspect of the present disclosure, systems and methods for using breath samples for detection of pathogens can include devices and materials for collecting breath samples, for creating a low-volume concentrated sample liquid, and for testing the sample liquid to ascertain the presence or absence of a pathogen of interest in the subject's breath. In one embodiment as shown in FIG. 1A and FIG. 1B, a collection device 100 for obtaining a breath sample from a subject can comprise a hollow tube 102 having a proximal end 104 for receiving the exhaled breath of a subject into the device. The collection device can further comprise a distal end 106 that includes a filter holding element 108 configured to hold a filter 110 in place during collection of a breath sample. For example, a subject can place the proximal end 104 of the tube 102 against or into their mouth and cough vigorously into the tube 102. Pulmonary aerosols containing the target virus travel distally through the tube 102 and are trapped in the filter 110 from which they can be extracted and transferred for an appropriate analysis technique.

In various embodiments, as shown by example in FIG. 1B, a filter 110 is situated at or near the distal end 106 of the tube 102. The filter 110 can be a porous filter comprising a filter material selected to capture and retain particles in a pulmonary aerosol. Filter materials include, but are not limited to, borosilicate glass, cotton, paper, acrylic, polypropylene, polytetrafluoroethylene (PTFE), and mixtures thereof. In some embodiments the filter material is present as fibers. In some embodiments, the filter comprises a woven filter material. In other embodiments, the filter comprises a non-woven filter material. In some embodiments, the filter material and the structure of the filter are selected to provide a porosity that provides capture of particles of a selected size or greater. In an embodiment, the filter has a pore size from about 1 μm to about 5 μm. In an aspect, the filter material can be selected to effectively capture potentially pathogenic particles in such a way that the sampled particles can be readily extracted from the filter for analysis. For example, filter material having a minimum level of hydrophobicity may be useful for using an aqueous buffer to extract the sample. In some embodiments, the filter material is treated to provide a particular level of hydrophobicity, e.g., the material can be coated with a coating to increase its hydrophobicity.

In some embodiments the filter holding element is configured to hold a replaceable filter that is shaped so as to be readily inserted and removed. For example, the filter can have a flattened shape, such as the disk-shaped filter 110 shown in FIG. 1A and FIG. 1B, and the filter holding element 108 can be shaped to hold it in place. As shown in FIG. 1A and FIG. 1B, the filter holding element 108 can include a lateral opening 112 through which a filter can be slid into or out of the filter holding element 108. The filter holding element 108 can further include a filter retention element 114 to maintain the filter in a particular position and prevent accidental removal during collection. For example, in some embodiments the filter retention element can include a hook or a latch. In particular embodiments, the filter retention element is flexible or articulated so that it can be disengaged from the filter to allow repositioning or removal of the filter.

The dimensions of the filter can be selected to effectively capture a breath sample delivered into the collection device. In an aspect, the surface area of the filter is such that it spans the diameter of the tube at the distal end. In some embodiments, the filter has a surface area from about 500 mm² to about 1000 mm². In more particular embodiments, the surface area can be from about 500 mm² to about 700 mm², or from about 600 mm² to about 800 mm². In some embodiments, the filter is a circular filter having a diameter from about 15 mm to about 50 mm. In more particular embodiments, the diameter can be from about 25 mm to about 50 mm, from about 25 mm to about 35 mm, from about 30 mm to about 40 mm, from about 35 mm to about 45 mm, or from about 40 mm to about 50 mm. In some embodiments, the filter has a thickness from about 0.5 mm to about 3.5 mm. In a particular embodiment, the filter thickness is from about 1.5 mm to about 3.5 mm, or from about 2 mm to about 3 mm.

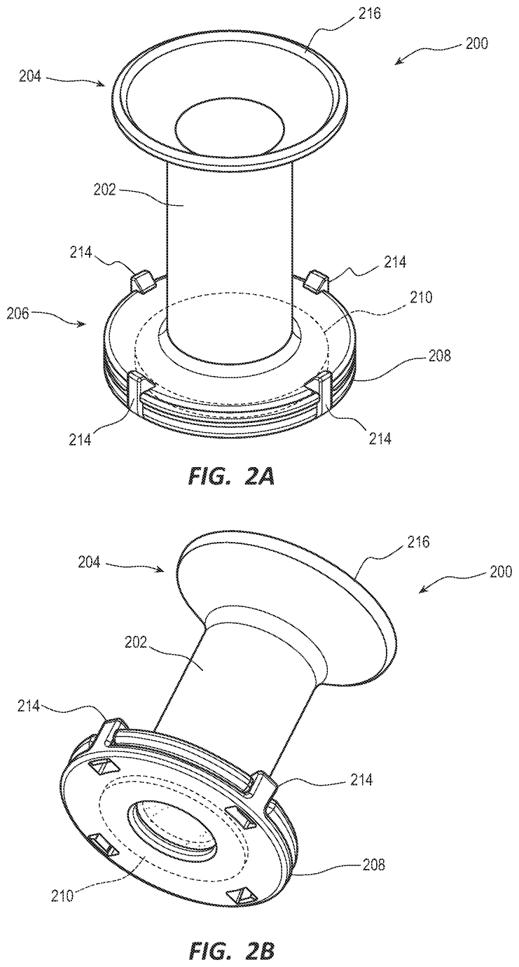

FIG. 2A and FIG. 2B show a breath sample collection device 200 with a filter holding element 208 according to another embodiment. As illustrated, a filter holding element 208 can be removably attached to the distal end 206 of the tube 202 such that removal of the filter holding element 208 allows placement/replacement of a filter 210. Once the filter 210 is placed over the distal end 206 or in the filter holding element 208, the filter holding element 208 can be reattached to the distal end 206. As illustrated in FIG. 2A and FIG. 2B, the filter holding element 208 can include one or more filter retention elements 214 that serve to maintain the position of the filter 210 and the filter holding element 208 during collection. In particular embodiments, one or more filter retention elements 214 can be flexible or articulated so that the filter holding element can be disengaged from the distal end.

As noted above, the proximal end of the tube can be configured for engagement with a subject's mouth to facilitate collection of a breath sample. In an aspect, such engagement provides for a cough or other exhalation to be delivered into the tube in a volume sufficient to produce a testable sample. More particularly, the proximal end can provide for creation of a sufficient seal between the subject's mouth and the collection device, so that a forceful breath or cough can be delivered into the device with minimal escape of breath around the proximal end. In some embodiments, the tube and the proximal end are shaped to allow insertion of at least the proximal end into a subject's mouth. In more particular embodiments, the tube and proximal end are shaped to allow insertion of most or all of the tube's length into the subject's mouth, where further insertion is avoided by a feature of the distal end, for example the filter holding element.

The tube can have a length and a diameter selected in accordance with any of the foregoing aspects, as well as other considerations based on intended use. For example, a shorter tube length may be selected to provide a more complete insertion into the subject's mouth or to provide a shorter path for exhaled particles to travel until contacting the filter. In another aspect, the tube may have an inner diameter large enough to not present an amount of resistance to the flow of the expelled breath that would result in leakage or filter displacement, and yet have an outer diameter that is not too large to fit into the subject's mouth. The age of the subject can also be a consideration, where tubes having a smaller length and/or diameter may be indicated for use with young subjects. In some embodiments, the tube has a length from about 20 mm to about 150 mm. In particular embodiments, the length can be from about 40 mm to about 100 mm, from about 20 mm to about 50 mm, from about 40 mm to about 80 mm, from about 70 mm to about 120 mm, or from about 100 mm to about 150 mm. In some embodiments, the tube has an outer diameter from about 20 mm to about 40 mm. In particular embodiments, the outer diameter is from about 20 mm to about 30 mm, from about 25 mm to about 35 mm, or from about 30 mm to about 40 mm. In some embodiments, the tube has an inner diameter from about 10 mm to about 35 mm. In particular embodiments, the inner diameter is from about 10 mm to about 20 mm, from about 15 mm to about 25 mm, from about 20 mm to about 30 mm, or from about 25 mm to about 35 mm.

In some embodiments, as shown by example in FIG. 2A and FIG. 2B, the proximal end 204 of the tube 202 can include a mouthpiece 216. In certain embodiments, the mouthpiece 216 may flare to a larger diameter than that of the tube 202, for example, to provide an opening having a larger diameter than that of the tube 202. In some embodiments, the mouthpiece 216 can be shaped for uses in which the proximal end 204 is inserted into the subject's mouth. In such embodiments, the mouthpiece 216 can aid retention of the proximal end 204 in the mouth during forceful breath or coughing. In some embodiments, the mouthpiece 216 can be shaped for uses in which the proximal end 204 is pressed against the subject's lips while remaining outside the subject's mouth. By way of nonlimiting example, the mouthpiece 216 may have a conical or a bell shape. In certain embodiments, the mouthpiece 216 can comprise a flexible material, such as rubber or a flexible plastic, that allows the mouthpiece 216 to conform to the subject's face to a degree and thereby create a seal to hinder escape of expelled breath around the proximal end 204.

The collection device as a whole or individual parts thereof can comprise materials selected to provide properties or performance in accordance with its intended use. Materials include, without limitation, plastics, glass, rubbers, metals, and mixtures thereof. In some embodiments, the material is composed or treated to provide a smooth surface inside the device that substantially prevents sample material from sticking to the device.

Breath samples collected in accordance with the present disclosure can be tested for the presence of pathogens by a number of methods, including the use of assay systems such as lateral flow assays. The present disclosure describes materials and methods for lateral flow assay of breath samples to detect the presence of respiratory viruses and other pathogens. In various embodiments, materials and methods described herein are for detecting SARS-CoV-2. In an aspect, the materials and methods disclosed herein can provide stronger detection signals more rapidly, at least in part due to the ability to effectively employ smaller volume sample liquids in which potential analytes are more concentrated.

In accordance with the present disclosure, a method of detecting the presence of a pathogen in a breath sample can comprise collecting a breath sample from a subject and creating from said sample a sample liquid that can be tested for the pathogen. In some embodiments, a sample liquid can be created by collecting a breath sample using a collection device described herein and extracting the sample from the filter. In various embodiments, extraction can be done by irrigating or immersing the filter in a suitable liquid buffer, thereby transferring into the liquid the breath sample components captured by the filter. In some embodiments, extraction can be performed by running a volume of the liquid buffer through the tube of the collection device while the filter is still in place in the filter holding element. This may include rinsing the interior surface of the tube to capture any sample material that may have adhered thereto and add said material to the sample liquid.

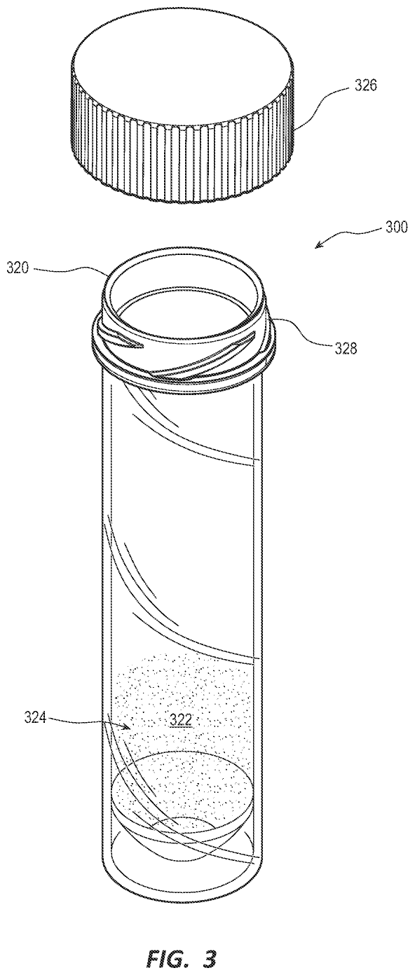

The sample liquid can then be further prepared for assay by adding an indicator selected to bind specifically to the pathogen of interest and thereby tag said pathogen, if present, for detection. In various embodiments, the indicator is provided in the form of an indicator conjugate comprising the indicator conjugated to a capture antibody that binds specifically to a protein present in the pathogen. References to “indicator(s)” herein are understood to encompass both such indicator conjugates and unconjugated indicators. In some embodiments, an indicator is added to the sample liquid by placing the sample liquid into a vial containing the indicator. In an embodiment, as shown in FIG. 3, a vial 300 can include a mouth 320 and an interior surface 322 on which the indicator 324 is immobilized. For example the surface 322 may be coated with a coating that includes the indicator 324. In a particular embodiment, the indicator 324 is present on the interior surface 322 in a substantially dried or dehydrated form. The vial 300 can optionally include a cap 326 that can be attached to the mouth 320 for sealing. As shown, in some embodiments the cap 326 can engage the mouth 320 via matching or complementary threads 328.

The liquid buffer and immobilized indicator are designed so that when the sample liquid is added to the vial, the indicator disassociates from the interior surface and enters the sample liquid. The indicator then binds to the pathogen of interest, if said pathogen is present in the sample liquid. Depending on the epitope and indicator binding characteristics, indicator-pathogen binding may be one-to-one or many indicators may bind to the pathogen. In some embodiments, a density of indicator on the interior surface and an amount of liquid buffer are selected so as to produce a particular concentration of indicator in the sample liquid. In particular embodiments, the concentration of indicator is from about 3×10⁹ particles/μL to about 3×10⁵ particles/μL.

The composition of the liquid buffer can also be selected to provide various functions appropriate to the pathogen of interest and the assay, including, but not limited to, buffering sample pH, minimizing non-specific binding, neutralizing interferents, and in the case of lateral flow assays, controlling flow speed. As will be understood by those skilled in the art having benefit of this disclosure, these can be accomplished with the use of various salts, surfactants, detergents, stabilizing agents, or blocking reagents. In certain embodiments, the liquid buffer is a phosphate buffered saline (PBS) that includes a surfactant. Suitable surfactants include, but are not limited to, nonionic surfactants such as poloxamer 407, polyethylene glycol hexadecyl ether (Brij 58), polysorbate 20, polysorbate 80, Triton X-100, and Triton X-114.

An indicator can be selected that provides an optical signal in an assay of choice. In various embodiments, the indicator produces a signal that can be read by eye (qualitative or semi-quantitative) or by an instrument (quantitative). For lateral flow assays, the indicator can be provided as a particle that is large enough to produce a strong signal per binding event while still flowing readily through the assay material. In some embodiments, the indicator has a particle size from about 20 nm to about 500 nm. In some embodiments, the indicator comprises a fluorophore that can be excited by radiation of a particular wavelength to emit a light signal of a different wavelength and having an intensity proportional to the concentration of the fluorophore. In a particular embodiment, the fluorophore emits light in the range from about 530 nanometers to about 570 nanometers. In another particular embodiment, the fluorophore emits light in the range from about 600 nanometers to about 700 nanometers. In some embodiments, the indicator comprises metal nanoshell particles in which a metal such as gold, copper, or silver forms a shell around a dielectric core. In a particular embodiment, the indicator comprises gold nanoshells.

As discussed above, the indicator can be conjugated to a protein that binds specifically to the pathogen of interest. In certain embodiments, the pathogen of interest is SARS-CoV-2, and the indicator is conjugated to a capture antibody specific for a SARS-CoV-2 protein. SARS-CoV-2 proteins include spike (S), membrane (M), nucleocapsid (N), envelope (E), and hemagglutinin esterase (HE). In a particular embodiment, the capture antibody is specific to SARS-CoV-2 spike protein. In another particular embodiment, the capture antibody is specific to SARS-CoV-2 nucleocapsid protein.

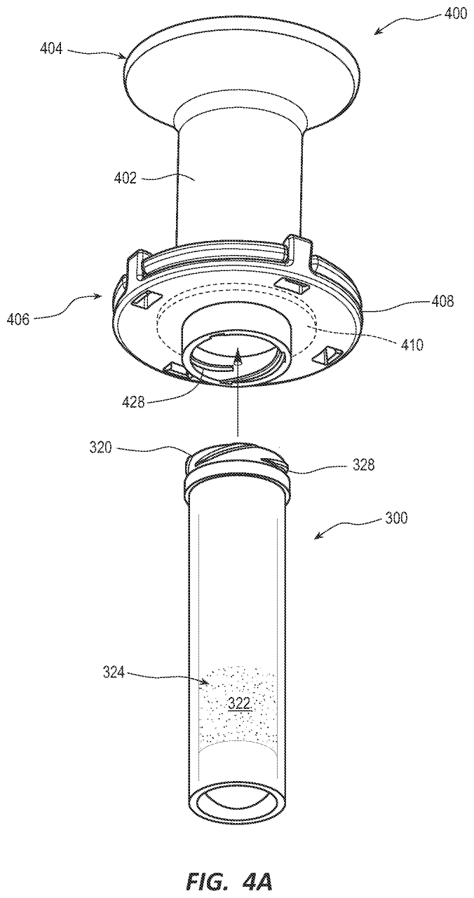

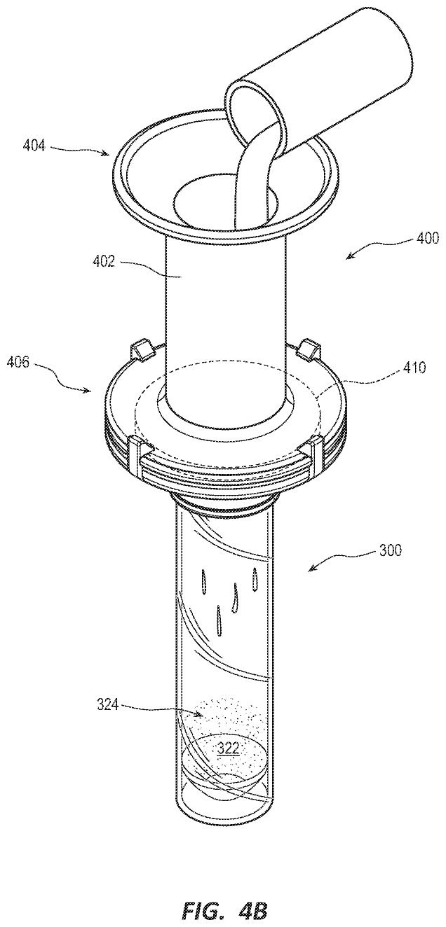

Creation of the sample liquid with indicator by the aforementioned steps can be facilitated by removably coupling the collection device to the vial. In some embodiments, the vial and collection device are configured for such coupling. FIG. 4A and FIG. 4B show an example of a collection device 400 that is configured for coupling its distal end 406 to the mouth 320 of a vial 300 with the filter holding element 408 and the filter 410 in place. In particular embodiments, one or both of the vial 300 and the collection device 400 include a coupling element by which coupling is facilitated. Coupling elements include but are not limited to threaded interfaces, clamps, clips, pin-and-socket interfaces. In an embodiment as shown in FIG. 4A, the mouth of the vial can comprise threads 328 that match or are complementary to threads 428 on the distal end 406 of the collection device 400.

As shown in FIG. 4B, once the vial 300 and collection device 400 are coupled, creation of the sample liquid can comprise introducing a liquid buffer into the proximal end 404 of the tube 402 so that the liquid buffer travels down the tube 402 to the distal end 406, through the filter 410, and into the vial 300. The indicator 324, which can be disposed on an inside surface 322 of the vial 300, then enters and is activated by the liquid, thus accomplishing creation of the sample liquid for testing. One aspect of using coupled devices for preparing the sample liquid is that a relatively small volume of liquid buffer can be used with a decreased risk of loss of sample liquid through leakage, spillage, or evaporation. In some embodiments, the amount of liquid buffer used is from about 40 μL to about 80 μL.

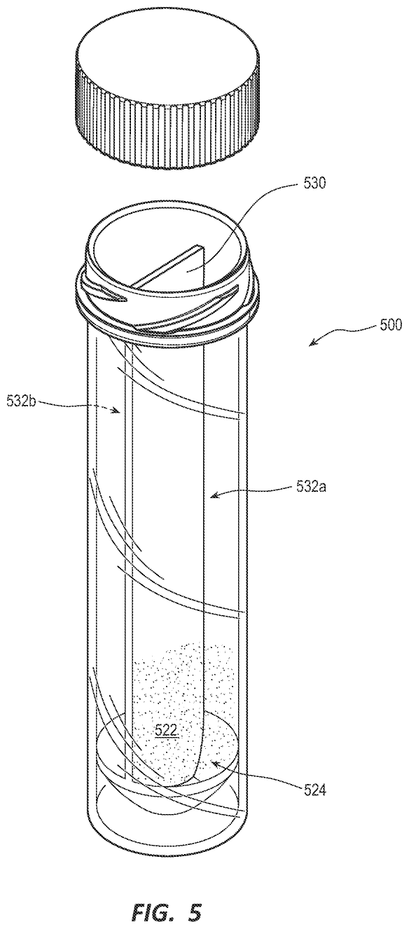

In another embodiment, as shown in FIG. 5, a vial 500 for use in creating a sample liquid can comprise a partition 530 situated within the vial 500 so that at least two interior spaces 532a, 532b are defined. The plurality of interior spaces can allow the vial 500 to serve more than one function in the process. For example, as shown in FIG. 5, a first interior space 532a can include an interior surface 522 onto which an indicator 524 is coated; and a separate or second interior space 532b is available for holding items or a substance, for example a reagent to be used in testing the sample liquid. In some embodiments, the second interior space 532b can contain a volume of liquid buffer for use in creating the sample liquid.

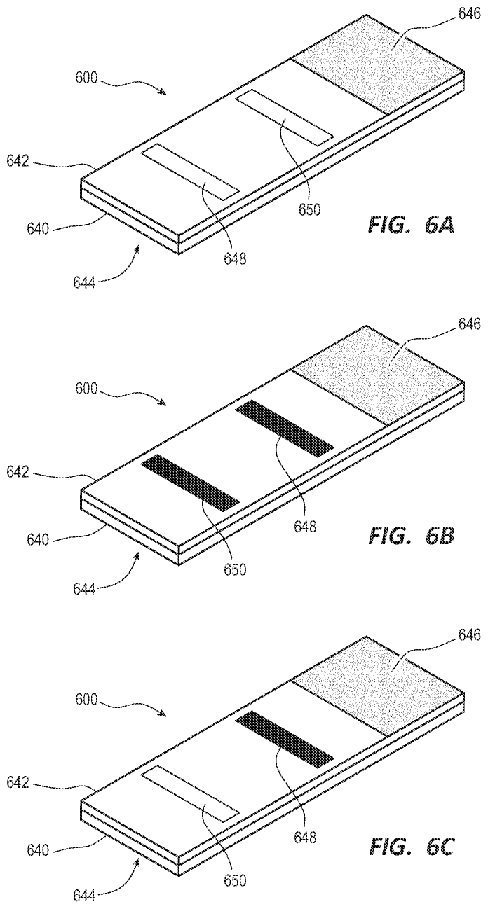

In accordance with the present disclosure, a method for detecting a pathogen in a breath sample can further comprise testing sample liquids prepared as described herein. In some embodiments, the sample liquids can be tested for the presence of a pathogen using a strip-based lateral flow assay. In an embodiment as shown in FIG. 6A, an assay strip 600 can comprise a support 640 on a portion of which a lateral flow layer 642 such as a nitrocellulose layer is situated. One end of the assay strip 600, the sample receiving end 644, includes the lateral flow layer 642, while the opposite end includes a wicking pad 646 comprising an absorbent material. The assay strip 600 further comprises a test zone 648 in which a test antibody is immobilized, where said test antibody binds specifically with a protein of the pathogen of interest for the assay. In certain embodiments, the test antibody is specific for a SARS-CoV-2 protein. In a particular embodiment, the test antibody is specific to SARS-CoV-2 spike protein. In another particular embodiment, the test antibody is specific to SARS-CoV-2 nucleocapsid protein. In some embodiments, the test antibody is specific to a different protein in the pathogen than the capture antibody.

In accordance with an embodiment, the assay strip 600 is arranged so that sample liquid applied to the sample receiving end 644 wicks up the strip through the lateral flow layer 642 (e.g., nitrocellulose layer) and into the wicking pad 646, which operates as a sink to maintain flow of the sample liquid in one general direction along the strip. The sample liquid encounters the test zone 648, where the test antibody binds a protein of the pathogen of interest, if said pathogen is present in the sample liquid. As discussed above, one or more indicator conjugates in the sample liquid are also bound to the pathogen of interest.

As shown in FIG. 6A, the assay strip 600 can further comprise a control zone 650 in which a protein with specific binding to a component of the indicator is immobilized. In certain embodiments, the indicator is an indicator conjugate comprising an indicator and a capture antibody and the control zone contains an immobilized antibody that is specific to the capture antibody. As such, the ligands in the control zone will bind unbound indicator conjugate regardless of whether pathogen is present in the sample liquid. In certain embodiments, the control zone is situated so that the sample liquid encounters it after passing through the test zone. Observing or measuring the presence of indicator in the control zone provides confirmation that sufficient sample liquid has traveled through the strip to enable detection, while observing the presence of indicator in the test zone indicates that the pathogen of interest is present in the sample. This principle is illustrated in FIG. 6B and FIG. 6C, each of which shows an assay strip 600 through which a sample liquid has run. In FIG. 6B a signal is observable in both the test zone 648 and the control zone 650, indicating a valid positive result. In FIG. 6C, a signal is observable in the control zone 650 only, indicating that the assay completed successfully but pathogen was not present in the sample, therefore providing a valid negative result.

Many current lateral flow assay strip formats include a sample pad for receiving a sample liquid and a conjugate pad containing indicator conjugate. In such strips, the sample pad serves to neutralize the sample liquid and filter out unwanted particulates, such as red blood cells in a blood sample. When the liquid reaches the conjugate pad, the indicator conjugate is released and mixes with the sample. However, these additional components increase the complexity of manufacturing these strips. Furthermore, due to the added material comprising these regions and the added strip length required to accommodate them, a larger volume of sample liquid should be passed through the strip in order to fully engage the test zone and provide a valid result. Creating a sample liquid to meet these requirements can result in a dilute analyte concentration, which in turn results in a signal that is too weak and/or slow to develop.

In contrast, assay strips according to the embodiments described herein do not include a sample pad or a conjugate pad. As discussed above, the present disclosure describes a sample liquid based on breath-borne aerosols and therefore is compositionally simpler than samples derived from mucus or saliva, with fewer components that can complicate handling and analysis. In addition, the sample liquid described herein can be prepared using smaller volumes while still including a sufficient potential analyte fraction to generate a strong assay signal. The sample liquid is also mixed with the indicator conjugate in the vial instead of on the test strip. Accordingly, in some embodiments, the assay strip can be shorter in length than strips that include a sample pad and/or conjugate pad. In particular embodiments, the length is from about 25 mm to about 45 mm. In some embodiments, the assay strip can have a decreased width. In particular embodiments, the width is from about 3 mm to about 7 mm, or from about 4 mm to about 5 mm.

In an aspect, the assay strips of the present disclosure can provide a signal indicating the presence or absence of a pathogen in a sample using a relatively small volume of sample liquid. In another aspect, the assay strips of the present disclosure can provide a signal indicating the presence or absence of a pathogen in a sample within a short time after the sample liquid is applied to the strip. In some embodiments, the time is less than about 5 minutes. In particular embodiments, the time is from about 10 seconds to about 90 seconds, or from about 15 seconds to about 60 seconds. In another aspect, the assay strips of the present disclosure provide enhanced sensitivity. In some embodiments, the detection limit of the assay per ml of sample liquid is from about 1 ng to about 100 ng. In an aspect, sensitivity can be determined by selection of the concentration of indicator in the sample liquid and the density of test antibodies in the test zone. In some embodiments, the test zone includes test antibodies at a density of from about 0.1 μg/mm to about 0.5 μg/mm.

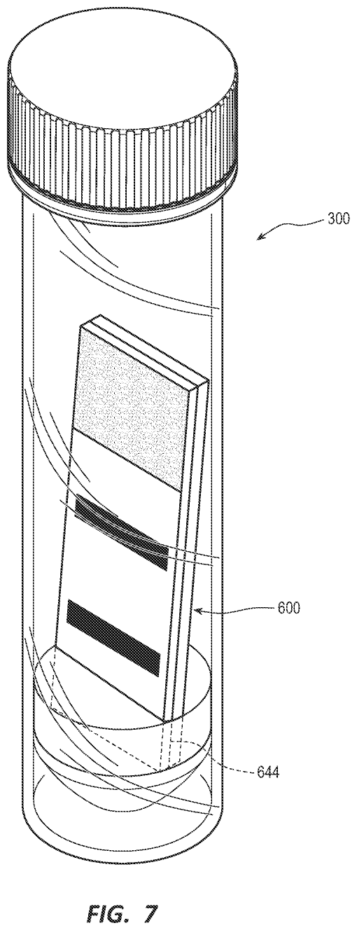

In some embodiments, contacting the sample liquid with the assay strip can comprise bringing the sample receiving end into contact with a volume of the sample liquid. In particular embodiments, an example of which is illustrated in FIG. 7, the assay strip 600 is placed into a vial 300 containing the sample liquid so that the sample receiving end 644 is in contact with the sample liquid.

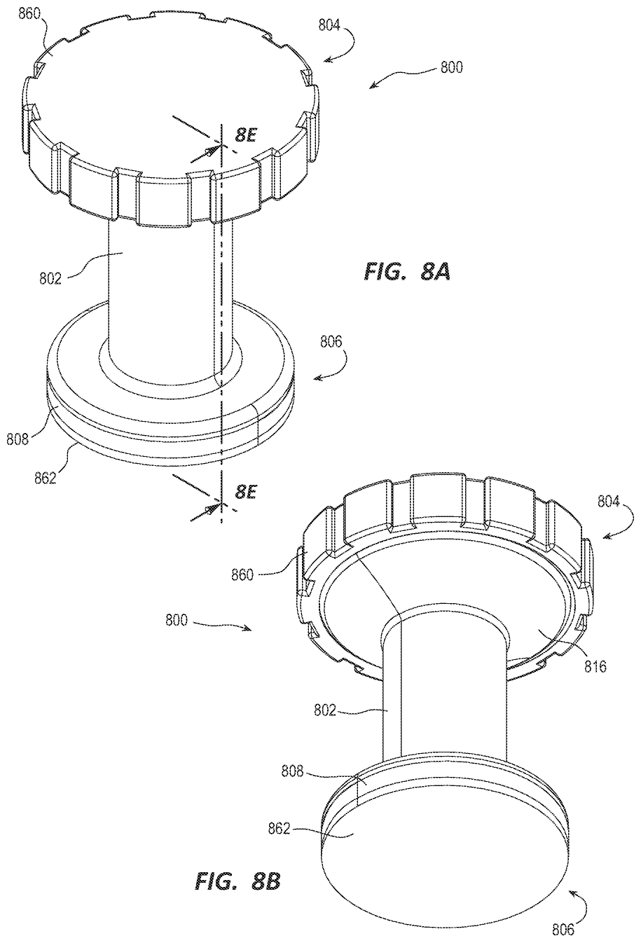

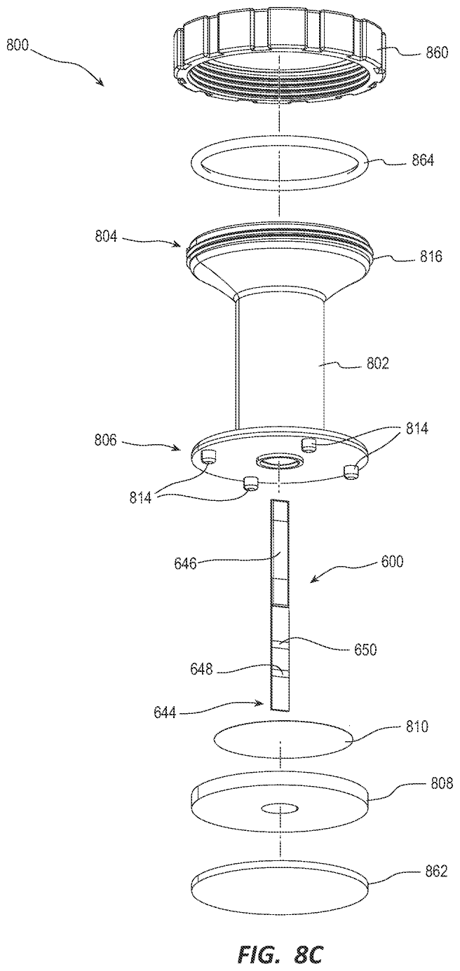

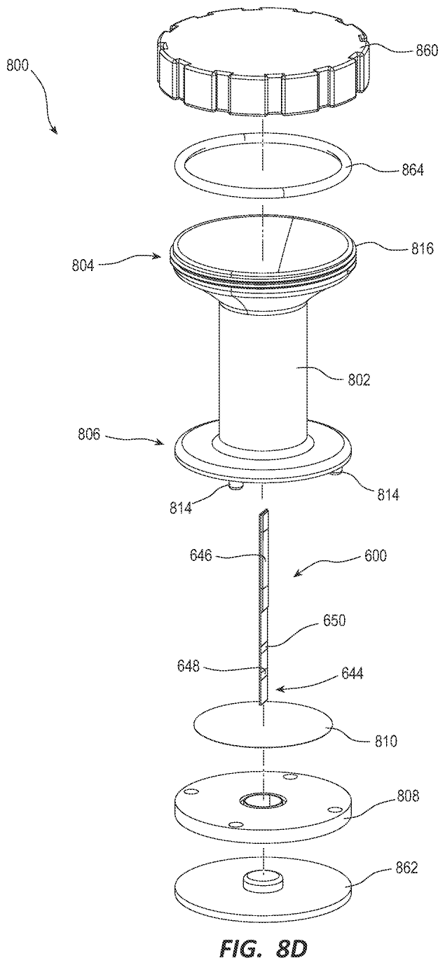

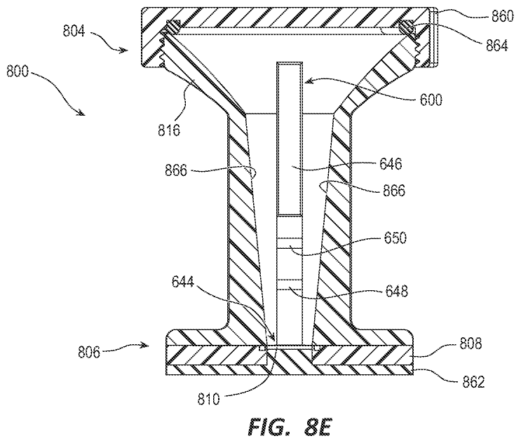

In some embodiments, a system for detecting a pathogen in a breath sample can comprise a strip-based assay provided in combination with a breath sample collection device adapted for use in both collecting and testing a breath sample. An example of such a system and use are illustrated in FIGS. 8A-8E. As shown in FIGS. 8A through 8E, a collection device 800 can comprise a hollow tube 802 having a proximal end 804 for receiving the exhaled breath of a subject into the device 800. As shown, in some embodiments the proximal end can include a mouthpiece 816. The collection device 800 can further comprise a distal end 806 that includes a filter holding element 808 configured to hold a filter 810 in place during collection of a breath sample. As illustrated in FIGS. 8C and 8D, the distal end 806 and/or the filter holding element 808 may include one or more filter retention elements 814 that serve to maintain the position of the filter 810 and the filter holding element 808 during collection.

The collection device 800 can be adapted to function as a container for materials used in sample testing. To this end, the device 800 can include a cap 860 configured to be removably secured to the proximal end 804. In some embodiments, as illustrated in FIGS. 8C and 8D, the cap 860 can be configured for securement to the proximal end 804 by a screw thread engagement. Other cap configurations include, but are not limited to, a snap-fit cap, a friction fit cap, a hinged flip cap, and a locking safety cap.

The collection device 800 can further include a plug 862 configured to be removably secured to the distal end 806. In some embodiments, the plug may be configured to engage a feature situated at the distal end 806—for example, the filter holder 808 as illustrated in FIGS. 8C-8E—to provide for securement. Such engagement may include, but is not limited to, snap fit engagement, screw thread engagement, and friction fit engagement.

These components may be configured so that the interior of the collection device 800 is rendered substantially fluid-tight when the cap 860 and plug 862 are secured in place. The term “fluid-tight” as used herein can include resistance to the passage of liquids and/or gases. In some embodiments, the collection device 800 can further include an O-ring 864 or seal to facilitate creation of a substantially fluid-tight seal between the cap 860 and the proximal end 804.

As noted above, such a collection device may be configured to enclose an assay strip such as those described herein. As shown in FIGS. 8C-8E, the tube 802 may be configured so that an assay strip 600 fits within the tube 802 and is fully enclosed within the collection device 800 when the cap 860 and plug 862 are in place. Particularly, the tube 802 and assay strip 600 can be configured to allow the assay strip 600 to rest within the tube 802 so that a part of the strip, e.g., the sample receiving end 644, contacts the filter 810, as shown in FIG. 8E. In some embodiments, the tube 802 is configured so that at least part of the assay strip 600 can be seen while the assay strip 600 is fully enclosed within the collection device 800. In an embodiment, for example, at least a portion of the tube 802 is transparent.

A system such as described above and illustrated in FIGS. 8A-8E can be used in a method of detecting the presence of a pathogen in a breath sample. In some embodiments, when a user is provided with the collection device 800 having an assay strip 600 enclosed within, the method can comprise removing the cap 860 from the proximal end 804 and removing the assay strip 600 from the interior of the tube 802. The method can further comprise removing the plug 862 from the distal end 806. The method further comprises using the collection device 800 to collect a breath sample as described above, e.g., by directing a cough or other exhalation into the tube 802 via the mouthpiece 816.

A small amount of a liquid buffer can then be introduced into the collection device 800. In some embodiments the amount of liquid buffer is from about 40 μL to about 80 μL. In some embodiments, the amount of liquid buffer is delivered in a dropwise fashion and may comprise a selected number of drops, such as from one to five drops, or one to three drops, or two to three drops.

The amount of liquid buffer may be introduced into the collection device 800 in a manner so that the liquid buffer is brought into contact with breathed sample material and a sample liquid is formed. This can comprise manipulating the collection device 800 so that the liquid buffer contacts surfaces within the collection device 800 onto which the breathed sample material may have collected, particularly the filter 810 and, optionally, an interior surface 866 of the tube 802. Such manipulation may include shaking, swirling, or inverting the collection device 800, or any combination of these actions. The cap 860 and/or the plug 862 may be replaced beforehand in order to prevent potential loss and/or contamination of the sample material.

As described above, formation of the sample liquid can also comprise introducing into the sample liquid a conjugate comprising an indicator. In some embodiments, the conjugate may be included in the liquid buffer. In some embodiments, the conjugate may be immobilized on a surface inside the collection device 800, e.g., the interior surface 866 of the tube and/or the material of the filter 810, so that when the liquid buffer is added to the vial, the conjugate disassociates from said surface and enters the liquid buffer.

The method can further comprise allowing the sample liquid to collect on the filter 810 before testing. In some embodiments, the interior surface 866 of the tube 802 may be sloped or otherwise configured to funnel sample liquid toward the filter 810. Then the cap 860—if secured to the proximal end 804—is removed and the assay strip 600 is placed inside the tube 802 so that the sample receiving end 644 contacts sample liquid contained in the filter 810. As described above, sample liquid that moves up the assay strip 600 toward the wicking pad 646 will encounter the test zone 648 and control zone 650 and produce a result which can be observed. In some embodiments, the collection device 800 allows the result to be observed without removing or otherwise directly handling the assay strip 600.

In accordance with the present disclosure, the devices and materials described herein can be provided as a kit for use in detecting a pathogen in a breath sample. In some embodiments, a kit comprises a collection device and optionally at least one filter. In some embodiments, a kit can comprise an indicator conjugate, which is more particularly provided in a vial having an interior surface on which the indicator conjugate is immobilized. In certain embodiments, the kit comprises a collection device and a vial that are configured to be coupled. In certain embodiments, the kit can further comprise a volume of a liquid buffer. In a particular embodiment, the liquid buffer can be provided in a vial having a partition situated therein so as to separate a first interior space including the interior surface onto which the conjugate is coated from a second interior space which includes the liquid buffer. In a particular embodiment, the kit can include a fluid transfer device such as a pipet or syringe for use in extracting a sample from a filter with the liquid buffer.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

References to approximations are made throughout this specification, such as by use of the terms “substantially” and “about.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration. All ranges also include both endpoints.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A system for detecting a pathogen in a breath sample, comprising:
a. a collection device comprising a tube extending between a proximal end and a distal end, wherein the distal end comprises:
i. a filter holding element; and
ii. a filter comprising a filter material;
b. a conjugate comprising an indicator conjugated to a capture antibody, wherein said capture antibody is specific to a first protein of a pathogen of interest; and
c. a vial having a mouth and an interior surface onto which the conjugate is coated.

2. The system of claim 1, further comprising an assay strip, said assay strip comprising:
a. a support bearing a lateral flow layer, wherein said lateral flow layer includes
i. a sample receiving end;
ii. a test zone having a test antibody immobilized therein; and
iii. a control zone having immobilized therein a control antibody that is specific to the capture antibody; and
b. a wicking pad comprising an absorbent material.

3. The system of claim 2, wherein the assay strip has a width from about 3 mm to about 7 mm.

4. (canceled)

5. The system of any of claim 1, wherein the test antibody is specific for the first protein.

6. The system of claim 1, wherein the test antibody is specific for a second protein of the pathogen of interest.

7-15. (canceled)

16. The system of claim 1, wherein the filter material is coated with a hydrophobic coating.

17-19. (canceled)

20. The system of claim 1, wherein the first protein is SARS-CoV-2 spike protein.

21. The system of claim 1, wherein the first protein is SARS-CoV-2 nucleocapsid protein.

22. The system of claim 1, wherein the distal end of the collection tube is configured to couple to the mouth of the vial so as to allow fluid communication between the collection tube and the vial via the filter.

23. The system of claim 1, further comprising a liquid buffer configured such that the conjugate enters the liquid buffer to create a sample solution upon addition of the liquid buffer to the vial.

24-26. (canceled)

27. The system of claim 1, wherein the vial comprises a partition situated therein so as to separate a first interior space including the interior surface onto which the conjugate is coated from a second interior space.

28. The system of claim 27, wherein the second interior space contains a liquid buffer configured such that the conjugate enters the liquid buffer to create a sample solution upon addition of the liquid buffer to the first interior space.

29-44. (canceled)

45. A method of detecting a pathogen in a breath sample, comprising:
a. collecting a sample of expired breath from a subject in a filter comprising a filter material;
b. extracting the sample from the filter using an amount of a liquid buffer to create a sample liquid;
c. adding to the sample liquid a conjugate comprising an indicator conjugated to a capture antibody, wherein said capture antibody is specific to a first protein of a pathogen of interest;
d. providing an assay strip comprising a test zone having a test antibody immobilized therein;
e. contacting the sample liquid to the assay strip so that the sample liquid moves through the assay strip to encounter the test zone; and
f. detecting presence of the pathogen in the sample by measuring presence of the conjugate in the test zone.

46. (canceled)

47. The method of claim 45, wherein the adding step comprises introducing the sample liquid into a vial having a surface on which the conjugate is immobilized, and wherein the contacting step comprises placing the assay strip into the vial so as to contact the sample liquid.

48-57. (canceled)

58. A system for detecting a pathogen in a breath sample, comprising:
a. a device comprising:
i. a tube extending between a proximal end and a distal end, wherein the distal end comprises a filter holding element;
ii a filter situated in the filter holding element;
iii a cap removably secured to the proximal end, and
iv a plug removably secured to the distal end;
b. an assay strip comprising:
i. a support bearing a lateral flow layer, wherein said lateral flow layer includes:
a sample receiving end;
a test zone having a test antibody immobilized therein; and
a control zone having a control antibody immobilized therein, wherein
the control antibody is specific to a capture antibody, wherein said capture antibody is specific to a first protein of a pathogen of interest, and the test antibody is specific to the first protein or a second protein of the pathogen of interest; and
ii. a wicking pad comprising an absorbent material, wherein the assay strip is situated within the tube and enclosed within the device by the cap.

59. The system of claim 58, wherein the assay strip is situated within the tube so that the sample receiving end is in contact with the filter.

60. The system of claim 58, wherein at least part of the assay strip is visible through a portion of the tube.

61-78. (canceled)

79. The system of claim 58, further comprising a conjugate in which an indicator is conjugated to the capture antibody.

80. The system of claim 79, wherein the conjugate is immobilized on a surface within the device.

81. The system of claim 79, wherein the conjugate is immobilized on the filter.

82-87. (canceled)
